(19) United States
(12) Patent Application Publication
(10) Pub. No.: US 2025/0009239 A1
(43) Pub. Date: Jan. 9, 2025

(54) BLOOD PRESSURE ESTIMATION DEVICE, BLOOD PRESSURE ESTIMATION METHOD, AND RECORDING MEDIUM

(71) Applicant: Nuvoton Technology Corporation Japan, Kyoto (JP)

(72) Inventors: Keisuke OKUNO, Osaka (JP); Kazuhiro MURATA, Hyogo (JP)

(21) Appl. No.: 18/888,879

(22) Filed: Sep. 18, 2024

Related U.S. Application Data
(63) Continuation of application No. PCT/JP2023/011648, filed on Mar. 23, 2023.

(30) Foreign Application Priority Data
Mar. 30, 2022 (JP) 2022-055153
Oct. 11, 2022 (JP) 2022-163397

(51) Int. Cl. A61B 5/021; A61B 5/00

(52) U.S. Cl. CPC A61B 5/02125; A61B 5/7267

(57) ABSTRACT

A blood pressure estimation device includes: a sensing module that obtains biological information of a target; a blood-pressure-estimation-model inference module that inputs the biological information of the target which has been obtained into a trained model generated through machine learning to infer a blood pressure value estimation model for the target; and a blood-pressure estimation module that estimates a blood pressure value of the target based on the biological information of the target which has been obtained and the blood pressure value estimation model for the target which has been inferred.

### CROSS REFERENCE TO RELATED APPLICATIONS

This is a continuation application of PCT International Application No. PCT/JP2023/011648 filed on Mar. 23, 2023, designating the United States of America, which is based on and claims priority of Japanese Patent Application No. 2022-055153 filed on Mar. 30, 2022 and Japanese Patent Application No. 2022-163397 filed on Oct. 11, 2022. The entire disclosures of the above-identified applications, including the specifications, drawings and claims are incorporated herein by reference in their entirety.

### FIELD

The present disclosure relates to a blood pressure estimation device, a blood pressure estimation method, and a recording medium.

### BACKGROUND

Patent Literature (PTL) 1 through PTL 3 disclose techniques for estimating a blood pressure value by substituting biological information, such as a pulse wave and the heart's electrical activity, into a predetermined estimation equation of estimating a blood pressure value.

### CITATION LIST

### Patent Literature

PTL 1: Japanese Unexamined Patent Application Publication No. H7-289526
PTL 2: Japanese Patent No. 4971041
PTL 3: Japanese Patent No. 5984088

### SUMMARY

### Technical Problem

However, since there are small differences in a correspondence between biological information, such as a pulse wave and the heart's electrical activity, and a blood pressure value, the estimation equation of estimating a blood pressure value using the biological information provides both accurate and inaccurate estimations depending on a person. In other words, it is difficult to accurately estimate blood pressure values of a large number of people using a single estimation equation.

In view of the above, the present disclosure provides a blood pressure estimation device, etc. which can accurately estimate a blood pressure value for each of individuals.

### Solution to Problem

A blood pressure estimation device according to the present disclosure includes: a sensing module that obtains biological information of a target; a blood-pressure-estimation-model inference module that inputs the biological information of the target which has been obtained into a trained model generated through machine learning to infer a blood pressure value estimation model for the target; and a blood-pressure estimation module that estimates a blood pressure value of the target based on the biological information of the target which has been obtained and the blood pressure value estimation model for the target which has been inferred.

A blood pressure estimation method according to the present disclosure is a blood pressure estimation method to be executed by a computer. The blood pressure estimation method includes: obtaining biological information of a target; inputting the biological information of the target which has been obtained into a trained model generated through machine learning, and inferring a blood pressure value estimation model for the target; and estimating a blood pressure value of the target based on the biological information of the target which has been obtained and the blood pressure value estimation model for the target which has been inferred.

A recording medium according to the present disclosure is a non-transitory computer-readable recording medium for use in a computer, the recording medium having recorded thereon a computer program for causing the computer to execute the above-described blood pressure estimation method.

Note that these comprehensive or specific aspects of the present disclosure may be implemented by a system, a method, an integrated circuit, a computer program, or a recording medium such as a computer-readable CD-ROM, or by an optional combination of the system, the method, the integrated circuit, the computer program, and the recording medium.

### Advantageous Effects

A blood pressure estimation device, etc. according to one aspect of the present disclosure can accurately estimate a blood pressure value for each of individuals.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a block diagram illustrating one example of a blood pressure estimation device according to Embodiment 1.

FIG. 2 is a diagram illustrating specific examples of features.

FIG. 3 is a flowchart illustrating one example of operations carried out by the blood pressure estimation device according to Embodiment 1 when blood pressure is estimated.

FIG. 4 is a flowchart illustrating one example of operations carried out by the blood pressure estimation device according to Embodiment 1 when learning is performed.

FIG. 5 is a diagram illustrating a learning module according to Embodiment 1.

FIG. 6 is a block diagram illustrating one example of a blood pressure estimation device according to a variation of Embodiment 1.

FIG. 7 is a block diagram illustrating one example of a blood pressure estimation device according to Embodiment 2.

FIG. 8 is a diagram illustrating one example of operations carried out by the blood pressure estimation device according to Embodiment 2 when learning is performed.

FIG. 9 is a diagram illustrating one example of operations carried out by the blood pressure estimation device according to Embodiment 2 when additional learning is performed.

FIG. 10 is a diagram illustrating one example of operations carried out by the blood pressure estimation device according to Embodiment 2 when blood pressure is estimated.

FIG. 11 is a diagram illustrating another example of operations carried out by the blood pressure estimation device according to Embodiment 2 when blood pressure is estimated.

FIG. 12 is a diagram illustrating one example of operations carried out by a blood pressure estimation device according to Embodiment 3 when learning is performed.

### DESCRIPTION OF EMBODIMENTS

### (Circumstances Leading to an Aspect of the Present Disclosure)

Blood pressure values have been conventionally measured by a method using a cuff. However, although the accuracy is high, the method using a cuff places strain on a subject because the cuff applies pressure to the arm of the subject. Moreover, this method using a cuff is not suitable for successive blood pressure measurements.

Meanwhile, there is a method of estimating blood pressure values without using a cuff. This method is, specifically, a method of estimating a blood pressure value using biological information, such as the heart's electrical activity and a pulse wave. More specifically, it is a method of estimating a blood pressure value from a pulse transmission time (PTT). This PTT is a delay time of a pulse-wave signal with respect to an electrocardiac signal. Since blood pressure correlates with a PTT, as it has been known that an increase in blood pressure tends to reduce a PTT and a reduction in blood pressure tends to increase a PTT, a blood pressure value can be estimated using the heart's electrical activity and a pulse wave. For example, as disclosed by PTL 1 through PTL 3, various estimation equations for estimating a blood pressure value using biological information, such as a pulse wave and the heart's electrical activity, have been disclosed.

However, since there are small differences in a correspondence between biological information, such as a pulse wave and the heart's electrical activity, and a blood pressure value depending on a person, it is difficult to accurately estimate blood pressure values of a large number of people using a single estimation equation. It is conceivable that an estimation equation is selected in accordance with a subject from among a plurality of estimation equations prepared in advance. However, the selected estimation equation is not an estimation equation prepared for the subject, and thus may not be the most suitable estimation equation for the subject. Accordingly, it may be difficult to accurately estimate a blood pressure value of the subject.

In view of the above, a blood pressure estimation device, etc. which can accurately estimate a blood pressure value for each of individuals will be hereinafter described.

A blood pressure estimation device according to one aspect of the present disclosure includes: a sensing module that obtains biological information of a target; a blood-pressure-estimation-model inference module that inputs the biological information of the target which has been obtained into a trained model generated through machine learning to infer a blood pressure value estimation model for the target; and a blood-pressure estimation module that estimates a blood pressure value of the target based on the biological information of the target which has been obtained and the blood pressure value estimation model for the target which has been inferred.

According to the above, a trained model generated through machine learning is used to infer a blood pressure value estimation model for a target. This estimation model is a model for the target which is inferred from biological information of the target, and is the most suitable model for the target. For this reason, a blood pressure value can be accurately estimated for each of individuals using an inferred estimation model. Moreover, since a cuff is not used, the blood pressure estimation device can be downsized. For example, a wearable device can be equipped with a blood pressure measurement function. This downsizing can also improve portability of the blood pressure estimation device. In addition, since an estimation (measurement) of blood pressure without using a cuff improves the portability of a blood pressure estimation device, blood pressure can be measured successively or all the time. Accordingly, it is possible to grasp the condition of a target from an instant change in the blood pressure which had been conventionally difficult to determine.

For example, the blood pressure estimation device may further include a learning module that performs the machine learning based on items of biological information of a plurality of people to generate the trained model.

As described above, items of biological information of a plurality of people can be used to generate the trained model.

For example, the learning module may perform the machine learning based on blood pressure values and the items of biological information of the plurality of people to generate the trained model.

Since blood pressure values and items of biological information are used when a trained model is generated, the generated trained model can be additionally trained using biological information and a blood pressure value of a target.

For example, the learning module may perform the machine learning using the items of biological information of the plurality of people as input data and the blood pressure values of the plurality of people as training data, to generate the trained model.

Since machine learning is performed using items of biological information as input data and blood pressure values as training data when a trained model is generated, the generated trained model can be additionally trained using biological information of a target as input data and a blood pressure value of the target as training data.

For example, the learning module may: for each person in the plurality of people, generate a blood pressure value estimation model for the person, based on the blood pressure value of the person and the biological information of the person, and perform the machine learning using the items of biological information of the plurality of people as input data and the blood pressure value estimation models for the plurality of people which have been generated and the blood pressure values of the plurality of people as training data, to generate the trained model.

According to the above, for each person in a plurality of people, a blood pressure value estimation model can be generated from a relationship between a blood pressure value and biological information of the person. Then, machine learning performed using items of biological information of the plurality of people as input data and estimation models and blood pressure values of the plurality of people as training data can generate a trained model that uses biological information as an input to output an estimation model. As described, since blood pressure values of the plurality of people are used as training data and machine learning is performed in advance so as to reduce differences between estimated blood pressure values of the plurality of people estimated by the estimation models and the blood pressure values used as training data, additional learning using biological information and a blood pressure value of a target can be performed.

For example, the learning module may perform additional machine learning using the biological information of the target which has been obtained as input data and a blood pressure value of the target as training data, to generate the trained model personalized for the target.

Since the trained model is a model trained using items of biological information, etc. of people different from a target, there may be a case where a blood pressure value cannot be accurately estimated using an estimation model output from the trained model depending on the target. In regard to the issue, additional machine learning performed using biological information of the target as input data and a blood pressure value of the target as training data can generate a trained model personalized for the target which outputs an estimation model that can accurately estimate a blood pressure value of the target.

For example, the blood-pressure-estimation-model inference module may input, into the trained model personalized for the target, items of biological information of the target each of which is the biological information of the target used when the additional machine learning was performed to infer a plurality of blood pressure value estimation models for the target each of which is the blood pressure value estimation model for the target, and may generate one estimation model personalized for the target from the plurality of blood pressure value estimation models for the target which have been inferred. The blood-pressure estimation module may estimate the blood pressure value of the target, based on the biological information of the target which has been obtained and the one estimation model personalized for the target.

According to the above, averaging of estimation models inferred using items of biological information of a target used when additional machine learning was performed can generate one estimation model personalized for the target. For example, an estimation model need not be inferred every time a blood pressure value of the target is estimated, and after an estimation model personalized for the target is generated, a blood pressure value of the target can be estimated using the estimation model personalized for the target.

For example, the learning module may: for each person in the plurality of people, generate a blood pressure value estimation model for the person, based on a blood pressure value of the person and the item of biological information of the person; and perform the machine learning based on the items of biological information of the plurality of people and the blood pressure value estimation models for the plurality of people which have been generated, to generate the trained model.

According to the above, for each person in a plurality of people, a blood pressure value estimation model can be generated from a relationship between a blood pressure value and biological information of the person. Then, machine learning performed using the items of biological information of the plurality of people as input data and the estimation models of the plurality of people as training data can generate a trained model that uses biological information as an input to output an estimation model.

For example, the learning module may generate the blood pressure value estimation models for the plurality of people through regression analysis.

According to the above, for each person in a plurality of people, a blood pressure value estimation model can be generated from a relationship between a blood pressure value and biological information of the person through regression analysis.

For example, the blood-pressure-estimation-model inference module may infer, as the blood pressure value estimation model for the target, a polynomial that uses the biological information of the target which has been obtained as a variable.

As described above, a blood pressure value estimation model of a target may be a polynomial.

For example, the blood-pressure-estimation-model inference module may infer, as the blood pressure value estimation model for the target, a weighting factor of a neural network that uses the biological information of the target which has been obtained as an input.

As described above, a blood pressure value estimation model of a target may be a neural network.

For example, the biological information may pertain to the heart's electrical activity and a pulse wave.

According to the above, the heart's electrical activity and a pulse wave correlate with blood pressure, a blood pressure value can be estimated based on biological information pertaining to the heart's electrical activity and a pulse wave.

For example, the blood-pressure-estimation-model inference module may input, as the biological information of the target which has been obtained, an electrocardiac waveform, a pulse waveform, and information for conforming a time period of the electrocardiac waveform to a time period of the pulse waveform into the trained model, to infer the blood pressure value estimation model for the target. The blood-pressure estimation module may estimate the blood pressure value of the target, based on a feature pertaining to the heart's electrical activity and the pulse wave as the biological information of the target which has been obtained and the blood pressure value estimation model for the target which has been inferred.

According to the above, the most suitable estimation model for a target can be inferred. In addition, when a blood pressure value of a target is estimated using an inferred estimation model, an electrocardiac waveform and a pulse waveform need not be used. Instead, a blood pressure value of the target can be simply estimated using features pertaining to the heart's electrical activity and a pulse wave.

For example, the information for conforming the time period of the electrocardiac waveform to the time period of the pulse waveform may include a pulse transmission time (PTTv) from an R wave of the electrocardiac waveform to a valley of the pulse waveform and a time period (peak-to-peak interval (PPI)) from a peak of the pulse waveform to a next peak of the pulse waveform.

According to the above, since a time period of an electrocardiac waveform of a target can conform to a time period of a pulse waveform of the target using a PTTv and a PPI, the most suitable estimation model for the target can be inferred.

For example, the blood-pressure estimation module may multiply the biological information of the target which has been obtained by a coefficient included in the blood pressure value estimation model for the target which has been inferred, to estimate the blood pressure value of the target.

A simple method as described above can estimate a blood pressure value.

A blood pressure estimation method according to one aspect of the present disclosure is a blood pressure estimation method to be executed by a computer. The blood pressure estimation method includes: obtaining biological information of a target; inputting the biological information of the target which has been obtained into a trained model generated through machine learning, and inferring a blood pressure value estimation model for the target; and estimating a blood pressure value of the target based on the biological information of the target which has been obtained and the blood pressure value estimation model for the target which has been inferred.

According to the above, a blood pressure method that can accurately estimate a blood pressure value for each of individuals can be provided.

A recording medium according to one aspect of the present disclosure is a non-transitory computer-readable recording medium for use in a computer, the recording medium having recorded thereon a computer program for causing the computer to execute the above-described blood pressure estimation method.

According to the above, a recording medium that can accurately estimate a blood pressure value for each of individuals can be provided.

Hereinafter, embodiments will be described in detail with reference to the drawings.

Note that the embodiments below each describe a general or specific example. The numerical values, shapes, materials, elements, the arrangement and the connection of the elements, steps, orders of the steps, etc. illustrated in the following embodiments are mere examples, and thus are not intended to limit the present disclosure.

### Embodiment 1

Hereinafter, a blood pressure estimation device according to Embodiment 1 will be described.

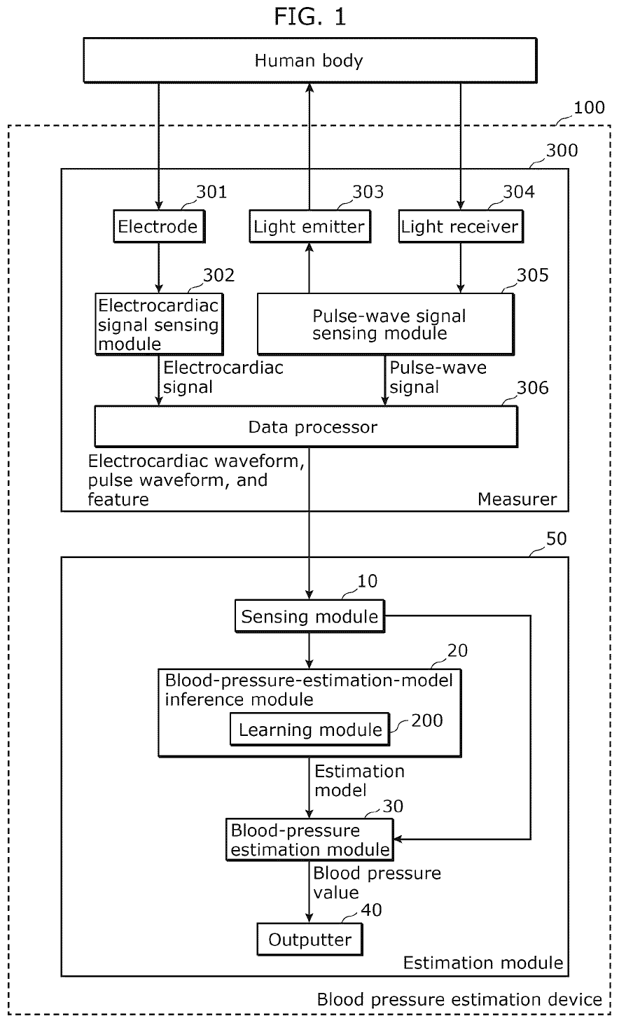

FIG. 1 is a block diagram illustrating one example of blood pressure estimation device 100 according to Embodiment 1.

Blood pressure estimation device 100 estimates a blood pressure value of a person. Hereinafter, a person whose blood pressure value is to be estimated is also called a target. Blood pressure estimation device 100 can estimate a blood pressure value by a method not using a cuff. For this reason, blood pressure estimation device 100 can be implemented as, for example, a wearable device.

Blood pressure estimation device 100 includes estimation module 50 and measurer 300.

Measurer 300 measures the heart's electrical activity and a pulse wave of a person. Measurer 300 includes electrode 301, electrocardiac signal sensing module 302, light emitter 303, light receiver 304, pulse-wave signal sensing module 305, and data processor 306.

Electrocardiac signal sensing module 302 obtains an electrocardiac signal via electrode 301 (specifically, two electrodes) brought into contact with a human body. Electrocardiac signal sensing module 302 includes, for example, an amplifier circuit, a filter circuit, and an analog-to-digital (AD) converter circuit. With this, a feeble electrocardiac signal is converted into a digital value after going through amplification and noise removal processes.

Pulse-wave signal sensing module 305 obtains a pulse-wave signal based on reflected light received by light receiver 304. The reflected light is light emitted from light emitter 303 and then reflected off a human body. For example, light receiver 304 converts an amount of the received reflected light into a voltage value, and outputs, as a pulse-wave signal, the voltage value to pulse-wave signal sensing module 305. Pulse-wave signal sensing module 305 includes, for example, an amplifier circuit, a filter circuit, and an analog-to-digital (AD) converter circuit. With this, a feeble pulse-wave signal is converted into a digital value after going through amplification and noise removal processes. Note that transmitted light transmitted through a human body may be used instead of reflected light reflected off the human body.

Data processor 306 extracts biological information from the electrocardiac signal and the pulse-wave signal obtained from electrocardiac signal sensing module 302 and pulse-wave signal sensing module 305, respectively. For example, the biological information pertains to the heart's electrical activity and a pulse wave, and is, specifically, an electrocardiac waveform and a pulse waveform, as well as various features.

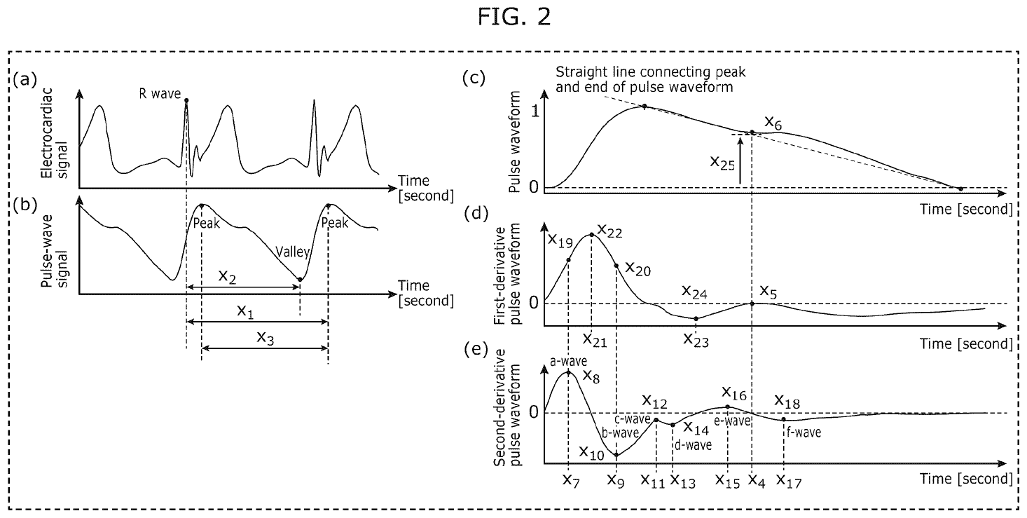

For example, data processor 306 extracts an electrocardiac waveform and a pulse waveform for one pulsation. Specifically, as for an electrocardiac waveform, data processor 306 detects R waves (see FIG. 2) and extracts, based on an interval between the detected R waves, an electrocardiac waveform for one pulsation. As for a pulse waveform, data processor 306 detects valleys (see FIG. 2) of a pulse wave and extracts, based on an interval between the detected valleys, a pulse waveform for one pulsation.

For example, data processor 306 extracts 25 types of features. Here, the 25 types of features will be described with reference to FIG. 2.

FIG. 2 is a diagram illustrating specific examples of the features. Part (a) of FIG. 2 is a diagram illustrating an electrocardiac signal, part (b) of FIG. 2 is a diagram illustrating a pulse-wave signal, part (c) of FIG. 2 is a diagram illustrating a pulse waveform for one pulsation, part (d) of FIG. 2 illustrates a first-derivative pulse waveform of the pulse waveform shown in part (c) of FIG. 2, and part (e) of FIG. 2 illustrates a second-derivative pulse waveform of the pulse waveform shown in part (c) of FIG. 2.

In FIG. 2, features x₁ through x₂₅ are shown as the 25 types of features.
Feature x₁ is a pulse transmission time (PTTp) from an R wave (a timepoint at which the heart contracts) of the electrocardiac waveform to a peak of the pulse waveform.
Feature x₂ is a pulse transmission time (PTTv) from the R wave of the electrocardiac waveform to a valley of the pulse waveform.
Feature x₃ is a time period (peak-to-peak interval (PPI)) from a peak of the pulse waveform to the next peak of the pulse waveform.
Feature x₄ is the time at which a value is zero between the e-wave (the last peak of the second-derivative pulse waveform) and the f-wave (the last valley of the second-derivative pulse waveform) of the second-derivative pulse waveform.
Feature x₅ is a value at the time x₄ in the first-derivative pulse waveform.
Feature x₆ is a value at the time x₄ in the pulse waveform.
Feature x₇ is the time of the a-wave (the first peak of the second-derivative pulse waveform) of the second-derivative pulse waveform.
Feature x₈ is a value at the time x₇ in the second-derivative pulse waveform.
Feature x₉ is the time of the b-wave (the first valley of the second-derivative pulse waveform) of the second-derivative pulse waveform.
Feature x₁₀ is a value at the time x₉ in the second-derivative pulse waveform.
Feature x₁₁ is the time of the c-wave (the second peak of the second-derivative pulse waveform) of the second-derivative pulse waveform.
Feature x₁₂ is a value at the time x₁₁ in the second-derivative pulse waveform.
Feature x₁₃ is the time of the d-wave (the second valley of the second-derivative pulse waveform) of the second-derivative pulse waveform.
Feature x₁₄ is a value at the time x₁₃ in the second-derivative pulse waveform.
Feature x₁₅ is the time of the e-wave of the second-derivative pulse waveform.
Feature x₁₆ is a value at the time x₁₅ in the second-derivative pulse waveform.
Feature x₁₇ is the time of the f-wave of the second-derivative pulse waveform.
Feature x₁₈ is a value at the time x₁₇ in the second-derivative pulse waveform.
Feature x₁₉ is a value at the time x₇ in the first-derivative pulse waveform.
Feature x₂₀ is a value at the time x₉ in the first-derivative pulse waveform.
Feature x₂₁ is the time of the peak of the first-derivative pulse waveform.
Feature x₂₂ is a value at the time x₂₁ in the first-derivative pulse waveform.
Feature x₂₃ is the time of the valley of the first-derivative pulse waveform.
Feature x₂₄ is a value at the time x₂₃ in the first-derivative pulse waveform.
Feature x₂₅ is a value at the time x₄ in the straight line connecting the peak and the end of the pulse waveform.

Note that electrocardiac waveforms and pulse waveforms for several pulsations may be extracted to extract the average electrocardiac waveform and the average pulse waveform for the several pulsations and the average of each of the features of the electrocardiac waveforms and the pulse waveforms for several pulsations.

Estimation module 50 estimates a blood pressure value of a target. Estimation module 50 includes sensing module 10, blood-pressure-estimation-model inference module 20, blood-pressure estimation module 30, and outputter 40. Blood pressure estimation device 100 (estimation module 50) is a computer including a processor, memory, etc. The memory is read-only memory (ROM), random-access memory (RAM), etc., and can store programs to be executed by the processor. Sensing module 10, blood-pressure-estimation-model inference module 20, blood-pressure estimation module 30, and outputter 40 are implemented by the processor or the like that executes programs stored in the memory.

Sensing module 10 obtains biological information of the target. Specifically, sensing module 10 obtains, from measurer 300, an electrocardiac waveform, a pulse waveform, and various features of the target.

Blood-pressure-estimation-model inference module 20 inputs the obtained biological information of the target into a trained model generated through machine learning to infer a blood pressure value estimation model for the target. For example, blood-pressure-estimation-model inference module 20 inputs, into the trained model, an electrocardiac waveform, a pulse waveform, and information for conforming a time period of the electrocardiac waveform to a time period of the pulse waveform as the obtained biological information of the target, to infer a blood pressure value estimation model for the target. The information for conforming a time period of the electrocardiac waveform to a time period of the pulse waveform includes, for example, a pulse transmission time (PTTv) from an R wave of the electrocardiac waveform to a valley of the pulse waveform and a time period (PPI) from a peak of the pulse waveform to a next peak of the pulse waveform. For example, blood-pressure-estimation-model inference module 20 infers the highest blood pressure value estimation model and the lowest blood pressure value estimation model as blood pressure value estimation models for the target. Note that blood-pressure-estimation-model inference module 20 may include learning module 200. Learning module 200 performs machine learning based on items of biological information of a plurality of people to generate a trained model.

Blood-pressure estimation module 30 estimates a blood pressure value of the target, based on the obtained biological information of the target and the inferred blood pressure value estimation model for the target. For example, blood-pressure estimation module 30 estimates a blood pressure value of the target, based on features pertaining to the heart's electrical activity and a pulse wave as the obtained biological information of the target and the inferred blood pressure value estimation model for the target. The features pertaining to the heart's electrical activity and a pulse wave are features extracted from an electrocardiac waveform and a pulse waveform, and are, more specifically, the above-described features x₁ through x₂₅. However, features to be used for an estimation of a blood pressure value of the target are non-limiting. The features may be some of features x₁ through x₂₅ or may be other different features.

Outputter 40 outputs an estimated blood pressure value. For example, outputter 40 outputs the estimated highest blood pressure value and the estimated lowest blood pressure value. For example, blood pressure estimation device 100 may include a display unit (e.g., a display) or a speech outputter (a loudspeaker), and may display an estimated blood pressure value or may output the estimated blood pressure value by voice.

Note that measurer 300 need not be an element to be included in blood pressure estimation device 100. For example, blood pressure estimation device 100 (estimation module 50) may obtain an electrocardiac waveform, a pulse waveform, and various features from measurer 300 provided separately from blood pressure estimation device 100.

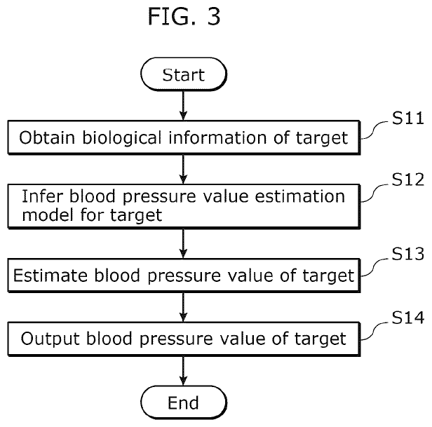

Next, details of operations carried out by blood pressure estimation device 100 will be described with reference to FIG. 3 through FIG. 5.

FIG. 3 is a flowchart illustrating one example of operations carried out by blood pressure estimation device 100 according to Embodiment 1 when blood pressure is estimated.

First, sensing module 10 obtains biological information of a target (step S11). As described above, sensing module 10 obtains, for example, an electrocardiac waveform, a pulse waveform, and various features (e.g., features x₁ through x₂₅) of the target.

Next, blood-pressure-estimation-model inference module 20 inputs the obtained biological information of the target into a trained model generated through machine learning to infer a blood pressure value estimation model for the target (step S12). For example, blood-pressure-estimation-model inference module 20 inputs, into the trained model, the electrocardiac waveform, the pulse waveform, a PTTv (feature x₂), and a PPI (feature x₃) of the target which have been obtained to infer the highest blood pressure value estimation model and the lowest blood pressure value estimation model for the target.

For example, blood-pressure-estimation-model inference module 20 infers, as blood pressure value estimation models (the highest blood pressure value estimation model and the lowest blood pressure value estimation models) for the target, polynomials that use the obtained biological information of the target as variables. For example, these variables are features pertaining to the heart's electrical activity and a pulse wave, and are, specifically, features extracted from an electrocardiac waveform and a pulse waveform. More specifically, the variables are the above-described features x₁ through x₂₅. However, these variables are not limited to features x₁ through x₂₅, and may be some of features x₁ through x₂₅ or may be other different features.

For example, the highest blood pressure value estimation model and the lowest blood pressure value estimation model may be expressed by polynomials shown below as Equation 1 and Equation 2, where aᵢ and bᵢ are coefficients, c and d are constants, and xᵢ is a feature.

$$\begin{matrix}[ Math . 1 ] & \\ \begin{matrix}Highest blood \\ pressure value\end{matrix} = {\sum }_{i = 1}^{25} {a}_{i} {x}_{i} + c & ( Equation 1 )\end{matrix}$$

$$\begin{matrix}[ Math . 2 ] & \\ \begin{matrix}Lowest blood \\ pressure value\end{matrix} = {\sum }_{i = 1}^{25} {b}_{i} {x}_{i} + d & ( Equation 2 )\end{matrix}$$

Next, blood-pressure estimation module 30 estimates a blood pressure value of the target, based on the obtained biological information of the target and the inferred blood pressure value estimation model for the target (step S13). For example, blood-pressure estimation module 30 estimates a blood pressure value of the target by multiplying the obtained biological information of the target by a coefficient included in the inferred blood pressure value estimation model for the target. For example, blood-pressure estimation module 30 multiplies the obtained features x₁ through x₂₅ by coefficient aᵢ and coefficient bᵢ shown in the above Equation 1 and Equation 2, respectively, to estimate the highest blood pressure value and the lowest blood pressure value of the target.

Outputter 40 then outputs the estimated blood pressure value of the target (step S14). For example, outputter 40 outputs, to a display or the like, the estimated highest blood pressure value and the estimated lowest blood pressure value of the target to cause the display or the like to display the estimated highest blood pressure value and the estimated lowest blood pressure value of the target.

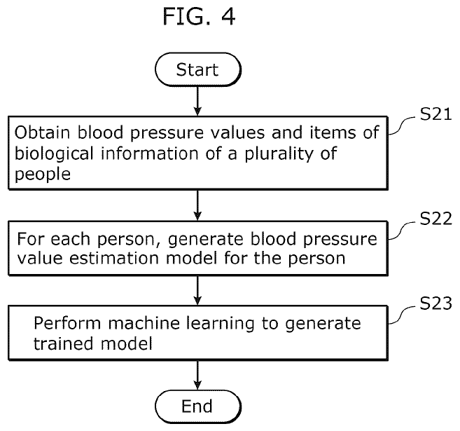

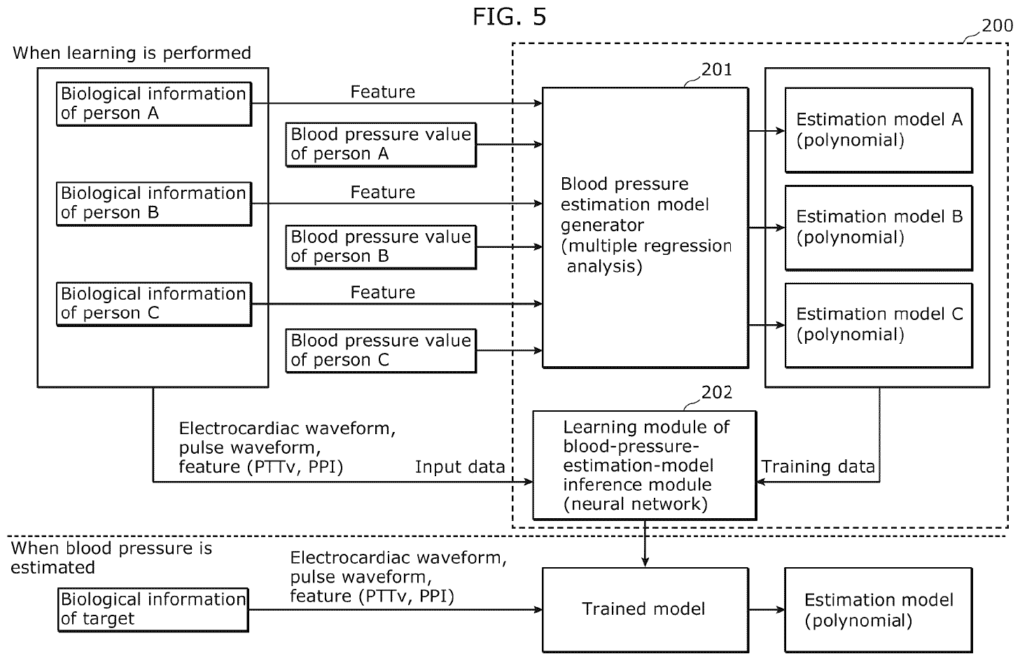

Next, details of operations carried out by blood pressure estimation device 100 when learning is performed by a trained model will be described with reference to FIG. 4 and FIG. 5.

FIG. 4 is a flowchart illustrating one example of operations carried out by blood pressure estimation device 100 according to Embodiment 1 when learning is performed.

FIG. 5 is a diagram illustrating learning module 200 according to Embodiment 1. The upper part of FIG. 5 shows blood pressure estimation model generator 201 and learning module of blood-pressure-estimation-model inference module 202 as functional elements to be used for learning performed by learning module 200. The lower part of FIG. 5 shows a schematic flow of inferencing an estimation model using a generated trained model.

First, learning module 200 obtains blood pressure values and items of biological information of a plurality of people (step S21). For example, learning module 200 obtains the highest blood pressure value and the lowest blood pressure value as blood pressure values of each person. The highest blood pressure value and the lowest blood pressure value are measured by, for example, a method using a cuff. In addition, learning module 200 obtains, as the biological information of each person, an electrocardiac waveform, a pulse waveform, and features pertaining to the heart's electrical activity and a pulse wave. For example, the features pertaining to the heart's electrical activity and the pulse wave are features extracted from the electrocardiac waveform and the pulse waveform, and are, specifically, the above-described features x₁ through x₂₅. Although FIG. 5 shows person A, person B, and person C as one example of the plurality of people to simplify description, blood pressure values and items of biological information of a large number of people may be obtained to be used for learning.

Next, for each person, learning module 200 (blood pressure estimation model generator 201) generates a blood pressure value estimation model for the person, based on the blood pressure value and the biological information of the person (step S22). For example, learning module 200 (blood pressure estimation model generator 201) generates these blood pressure value estimation models for the plurality of people through regression analysis. For example, a blood pressure value measured by a method using a cuff is used as training data and biological information (features) is used as input data to generate, for each of individuals, a blood pressure value estimation model for the individual through regression analysis. For example, as for person A, the highest blood pressure value of person A is used as training data and features x₁ through x₂₅ are used as input data to generate the highest blood pressure value estimation model for person A through multiple regression analysis. Also, the lowest blood pressure value of person A is used as training data and features x₁ through x₂₅ are used as input data to generate the lowest blood pressure value estimation model for person A through multiple regression analysis. In FIG. 5, the highest blood pressure value estimation model for person A and the lowest blood pressure value estimation model for person A are shown as estimation model A. Estimation model A is, for example, a polynomial using, as a variable, biological information (e.g., features x₁ through x₂₅) of person A. Likewise, estimation model B for person B (specifically, the highest blood pressure value estimation model for person B and the lowest blood pressure value estimation model for person B) and estimation model C for person C (specifically, the highest blood pressure value estimation model for person C and the lowest blood pressure value estimation model for person C) are generated.

As described above, an estimation model generated by blood pressure estimation model generator 201 through multiple regression analysis is a model for each individual, and features (input data) and a blood pressure value (training data) are prepared for each individual for the purpose of learning. An estimation model is a polynomial expressed by the above Equation 1 or Equation 2, but in some cases a coefficient may be determined to be zero and a term may be omitted during an estimation. Although a method of generating an estimation model by blood pressure estimation model generator 201 will be described later, it should be noted that this method of generating an estimation model by blood pressure estimation model generator 201 is not limited to a method using multiple regression analysis, and may be a method using other regression analyses such as a neural network. For example, the use of multiple regression analysis by blood pressure estimation model generator 201 can increase the processing speed. Meanwhile, the use of a neural network by blood pressure estimation model generator 201 can increase the accuracy of machine learning.

Next, learning module 200 (learning module of blood-pressure-estimation-model inference module 202) performs machine learning based on the obtained items of biological information of the plurality of people and the generated blood pressure value estimation models for the plurality of people to generate a trained model (step S23). For example, learning module of blood-pressure-estimation-model inference module 202 learns a neural network that outputs a blood pressure value estimation model based on the items of biological information of the plurality of people, to generate a trained neural network model (trained model).

Learning module of blood-pressure-estimation-model inference module 202 learns a neural network that uses, as input data, electrocardiac waveforms, pulse waveforms, PTTvs, and PPIs of the plurality of people (e.g., person A, person B, and person C) and, as training data, generated polynomials for the plurality of people (e.g., person A, person B, and person C) to output a blood pressure value estimation model (polynomial). In other words, leaner of blood-pressure-estimation-model inference module 202 performs machine learning based on the items of input data and the items of training data of the plurality of people to generate a neural network that outputs a coefficient (e.g., aᵢ or bᵢ) and a constant (e.g., c or d) for a polynomial. The use of the above-described trained neural network by blood-pressure-estimation-model inference module 20 enables blood-pressure-estimation-model inference module 20 to output, from an electrocardiac waveform, a pulse waveform, and features such as a PTTv and a PPI of an unidentified person (i.e., a target) not used for the learning, a coefficient and a constant for an estimation model suitable for the target, when blood pressure is estimated. Thereafter, a blood pressure value of the target can be estimated by multiplying the coefficient by a feature (e.g., features x₁ through x₂₅) of the target and adding the constant.

As has been described above, a trained model generated through machine learning is used to infer a blood pressure value estimation model for a target. This estimation model is a model inferred from biological information of the target, and thus is the most suitable model for the target. For this reason, a blood pressure value can be accurately estimated for each of individuals using an inferred estimation model. Moreover, since a cuff is not used, blood pressure estimation device 100 can be downsized. For example, a wearable device can be equipped with a blood pressure measurement function. This downsizing can also improve the portability of blood pressure estimation device 100. In addition, since an estimation (measurement) of blood pressure without using a cuff improves the portability of the blood pressure estimation device, blood pressure can be measured successively or all the time. Accordingly, it is possible to grasp the condition of a target from an instant change in the blood pressure which had been conventionally difficult to determine.

### Variation of Embodiment 1

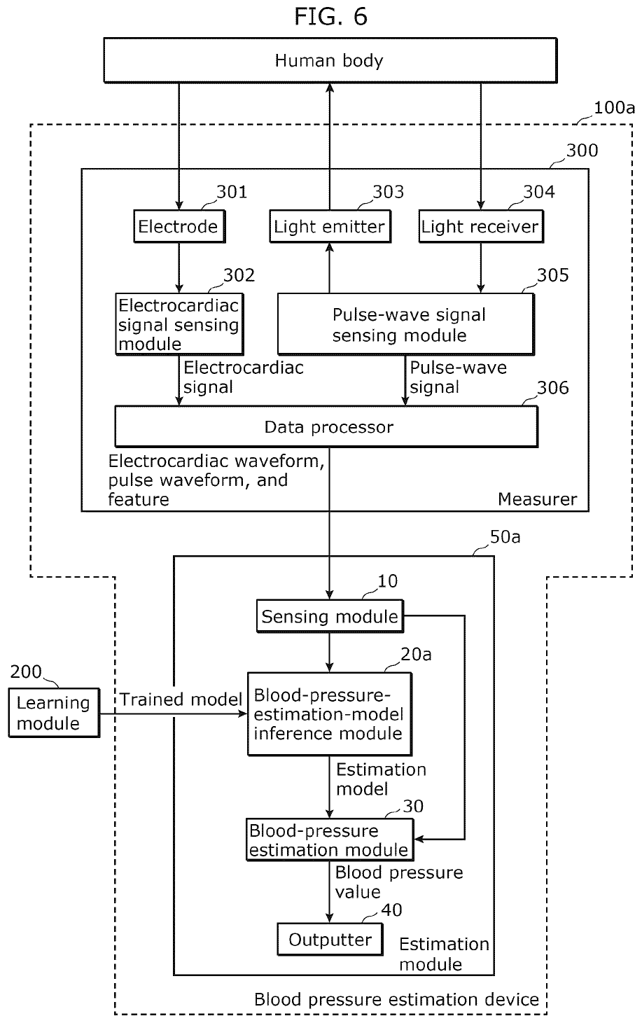

Although Embodiment 1 has presented an example in which blood-pressure-estimation-model inference module 20 includes learning module 200, blood-pressure-estimation-model inference module 20 need not include learning module 200. The foregoing will be described as a variation of Embodiment 1 with reference to FIG. 6.

FIG. 6 is a block diagram illustrating one example of blood pressure estimation device 100a according to the variation of Embodiment 1.

Blood pressure estimation device 100a according to the variation of Embodiment 1 is different from blood pressure estimation device 100 according to Embodiment 1, in that (i) blood pressure estimation device 100a includes estimation module 50a instead of estimation module 50 and (ii) blood-pressure-estimation-model inference module 20a of estimation module 50a does not include learning module 200. Other than the above, blood pressure estimation device 100a is basically the same as blood pressure estimation device 100 according to Embodiment 1. Accordingly, detailed description will be omitted.

For example, learning module 200 may be provided in an external computer outside blood pressure estimation device 100a. The external computer may be a server or the like. Learning module 200 provided in the external computer obtains items of biological information (electrocardiac waveforms, pulse waveforms, and features) of a plurality of people and blood pressure values (the highest blood pressure values and the lowest blood pressure values) of the plurality of people which are to be used as training data, to generate a trained model. Thereafter, blood pressure estimation device 100a may receive the trained model generated by learning module 200 via a communicator (not illustrated) used for communicating with the external computer, and blood-pressure-estimation-model inference module 20a may infer, using the received trained model, a blood pressure value estimation model for a target.

### Embodiment 2

Next, a blood pressure estimation device according to Embodiment 2 will be described.

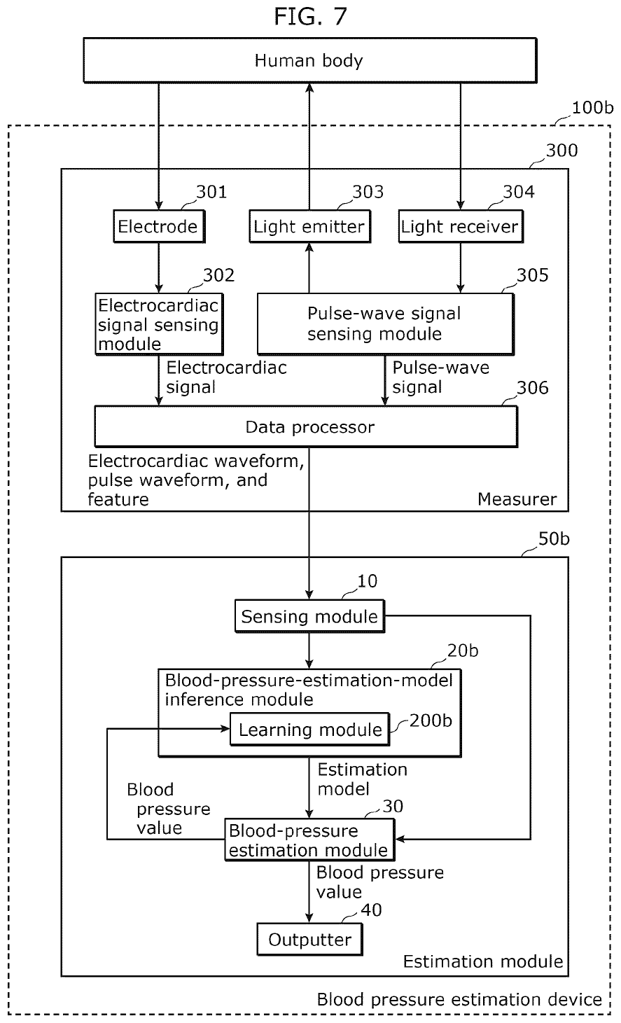

FIG. 7 is a block diagram illustrating one example of blood pressure estimation device 100b according to Embodiment 2.

Blood pressure estimation device 100b according to Embodiment 2 is different from blood pressure estimation device 100 according to Embodiment 1, in that blood pressure estimation device 100b includes estimation module 50b instead of estimation module 50. Estimation module 50b according to Embodiment 2 is different from estimation module 50 according to Embodiment 1, in that estimation module 50b includes blood-pressure-estimation-model inference module 20b instead of blood-pressure-estimation-model inference module 20. Blood-pressure-estimation-model inference module 20b is different from blood-pressure-estimation-model inference module 20 according to Embodiment 1, in that blood-pressure-estimation-model inference module 20b includes learning module 200b instead of learning module 200. Other than the above, blood pressure estimation device 100b is basically the same as blood pressure estimation device 100 according to Embodiment 1. Accordingly, detailed description will be omitted.

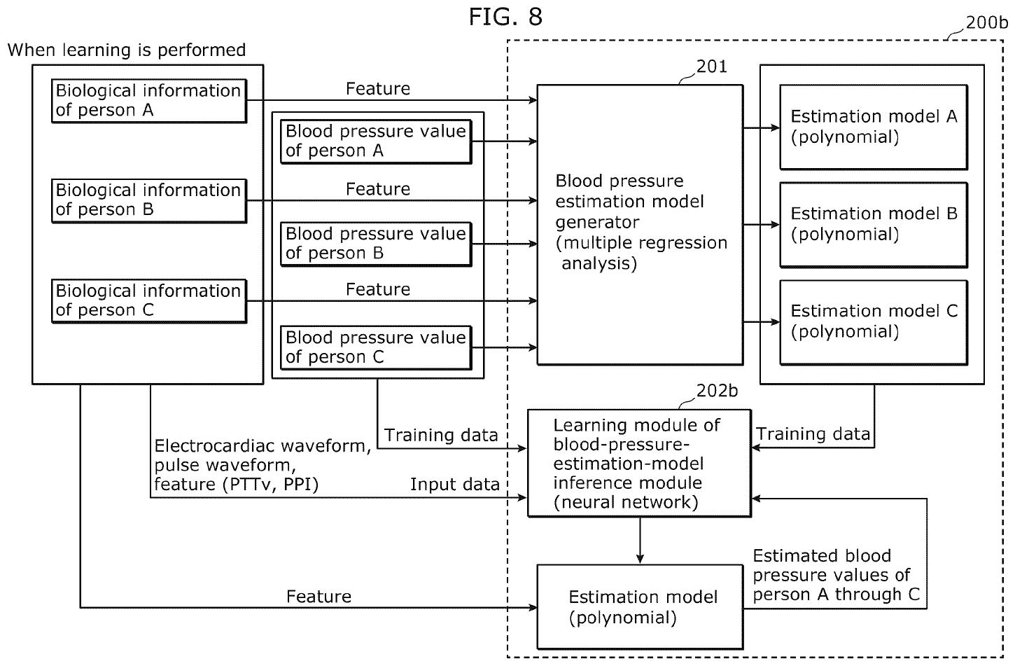

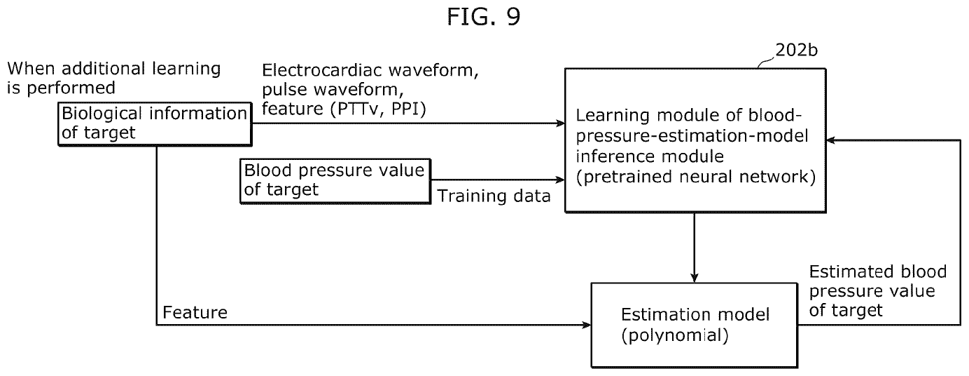

Learning module 200b performs machine learning based on blood pressure values and items of biological information of a plurality of people to generate a trained model. Learning module 200b also has a function of additionally training the trained model using a blood pressure value (e.g., the highest blood pressure value and the lowest blood pressure value measured by a method using a cuff) of a target and an estimated blood pressure value (e.g., the estimated highest blood pressure value and the estimated lowest blood pressure value) estimated by blood-pressure estimation module 30. Learning module 200b will be described in detail with reference to FIG. 8 and FIG. 9.

FIG. 8 is a diagram illustrating one example of operations carried out by blood pressure estimation device 100b according to Embodiment 2 when learning is performed.

First, learning module 200b obtains blood pressure values and items of biological information of a plurality of people. For example, learning module 200b obtains the highest blood pressure value and the lowest blood pressure value as blood pressure values of each person. The highest blood pressure value and the lowest blood pressure value are measured by, for example, a method using a cuff. In addition, learning module 200b obtains an electrocardiac waveform, a pulse waveform, and features pertaining to the heart's electrical activity and a pulse wave as the biological information of each person. For example, the features pertaining to the heart's electrical activity and the pulse wave are features extracted from the electrocardiac waveform and the pulse waveform, and are, specifically, the above-described features x₁ through x₂₅. Although FIG. 8 shows person A, person B, and person C as one example of the plurality of people to simplify description, blood pressure values and items of biological information of a large number of people may be obtained to be used for learning.

Next, for each person, learning module 200b (blood pressure estimation model generator 201) generates a blood pressure value estimation model for the person, based on the blood pressure value and the biological information of the person. For example, blood pressure estimation model generator 201 generates these blood pressure value estimation models for the plurality of people through regression analysis. For example, a blood pressure value measured by a method using a cuff is used as training data and biological information (features) is used as input data to generate, for each of individuals, a blood pressure value estimation model for the individual through regression analysis. For example, as for person A, the highest blood pressure value of person A is used as training data and features x₁ through x₂₅ are used as input data to generate the highest blood pressure value estimation model for person A through multiple regression analysis. Also, the lowest blood pressure value of person A is used as training data and features x₁ through x₂₅ are used as input data to generate the lowest blood pressure value estimation model for person A through multiple regression analysis. In FIG. 8, the highest blood pressure value estimation model for person A and the lowest blood pressure value estimation model for person A are shown as estimation model A. Estimation model A is, for example, a polynomial using, as a variable, biological information (e.g., features x₁ through x₂₅) of person A. Likewise, estimation model B for person B (specifically, the highest blood pressure value estimation model for person B and the lowest blood pressure value estimation model for person B) and estimation model C for person C (specifically, the highest blood pressure value estimation model for person C and the lowest blood pressure value estimation model for person C) are generated.

As described above, an estimation model generated by blood pressure estimation model generator 201 through multiple regression analysis is a model for each individual, and features (input data) and a blood pressure value (training data) are prepared for each individual for the purpose of learning. An estimation model is a polynomial expressed by the above Equation 1 or Equation 2, but in some cases a coefficient may be zero and a term may be omitted during an estimation. Although a method of generating an estimation model by blood pressure estimation model generator 201 will be described later, it should be noted that this method of generating an estimation model by blood pressure estimation model generator 201 is not limited to a method using multiple regression analysis, and may be a method using other regression analyses such as a neural network. For example, the use of multiple regression analysis by blood pressure estimation model generator 201 can increase the processing speed. Meanwhile, the use of a neural network by blood pressure estimation model generator 201 can increase the accuracy of machine learning.

Next, learning module 200b (learning module of blood-pressure-estimation-model inference module 202b) performs machine learning that uses, as input data, the items of biological information of the plurality of people and, as training data, the generated blood pressure value estimation models for the plurality of people and the blood pressure values of the plurality of people, to generate a trained model. For example, learning module of blood-pressure-estimation-model inference module 202b learns a neural network that outputs a blood pressure value estimation model based on the items of biological information of the plurality of people to generate a trained neural network model (trained model). In Embodiment 1, learning module of blood-pressure-estimation-model inference module 202 does not use blood pressure values of a plurality of people as training data, but in Embodiment 2, learning module of blood-pressure-estimation-model inference module 202b uses blood pressure values of the plurality of people training data.

Specifically, learning module of blood-pressure-estimation-model inference module 202b learns a neural network that uses, as input data, electrocardiac waveforms, pulse waveforms, PTTvs, and PPIs of the plurality of people (e.g., person A, person B, and person C) and, as training data, generated polynomials for the plurality of people (e.g., person A, person B, and person C) and blood pressure values of the plurality of people (e.g., person A, person B, and person C), to output a blood pressure value estimation model (polynomial). Every time learning module of blood-pressure-estimation-model inference module 202b performs learning, or stated differently, every time a weighting factor of the neutral network is updated, learning module of blood-pressure-estimation-model inference module 202b infers an estimation model based on the input data and, inputs features (e.g., features x₁ through x₂₅) into the inferred estimation model to estimate blood pressure values of the plurality of people (e.g., person A, person B, and person C). Learning module of blood-pressure-estimation-model inference module 202b compares the estimated blood pressure values with the blood pressure values that are the training data to obtain differences, and performs learning so as to reduce these differences. As described above, learning module of blood-pressure-estimation-model inference module 202b generates a trained model.

The use of the above-described trained neural network by blood-pressure-estimation-model inference module 20b enables blood-pressure-estimation-model inference module 20b to output, from an electrocardiac waveform, a pulse waveform, and features such as a PTTv and a PPI of an unidentified person (i.e., a target) not used for learning, a coefficient and a constant for an estimation model suitable for the target, when blood pressure is estimated. Thereafter, a blood pressure value of the target can be estimated by multiplying the coefficient by a feature (e.g., features x₁ through x₂₅) of the target and adding the constant. However, since the trained model is a model trained using items of biological information, etc. of people different from the target, there may be a case where a blood pressure value cannot be accurately estimated using an estimation model output from the trained model depending on the target.

In view of the above, additional learning by the trained model is performed using biological information, etc. of the target in Embodiment 2.

FIG. 9 is a diagram illustrating one example of operations carried out by blood pressure estimation device 100b according to Embodiment 2 when additional learning is performed.

Learning module 200b (learning module of blood-pressure-estimation-model inference module 202b) performs additional machine learning that uses obtained biological information of a target as input data and a blood pressure value of the target as training data to generate a trained model personalized for the target. Note that a blood pressure value of the target is used as training data when additional machine learning is performed, since it is difficult to prepare the correct estimation model for the target. For example, when the target uses blood pressure estimation device 100b for the first time, the blood pressure value and biological information (an electrocardiac waveform, a pulse waveform, etc.) are measured several times (e.g., three times) for learning module of blood-pressure-estimation-model inference module 202b to obtain data for additional learning. For example, the items of biological information are measured by measurer 300, and the blood pressure values are measured by a blood pressure monitor that uses a method using, for example, a cuff.

Learning module of blood-pressure-estimation-model inference module 202b uses, as input data, an electrocardiac waveform, a pulse waveform, a PTTv, and a PPI of the target and, as training data, a blood pressure value of the target to perform additional learning. In the same manner as prior learning, learning module of blood-pressure-estimation-model inference module 202b infers an estimation model, and inputs features (e.g., features x₁ through x₂₅) into the inferred estimation model to estimate a blood pressure value of the target. Learning module of blood-pressure-estimation-model inference module 202b compares the estimated blood pressure value with the blood pressure value that is the training data to obtain a difference, and performs learning so as to reduce the difference. With this, a trained model personalized for the target that outputs an estimation model that can accurately estimate a blood pressure value of the target can be generated.

As illustrated in FIG. 8, since the trained model has performed prior learning so as to reduce the differences between estimated blood pressure values and correct blood pressure values in Embodiment 2, additional learning by the trained model can be performed so as to reduce the difference between an estimated blood pressure value of the target and the correct blood pressure value of the target.

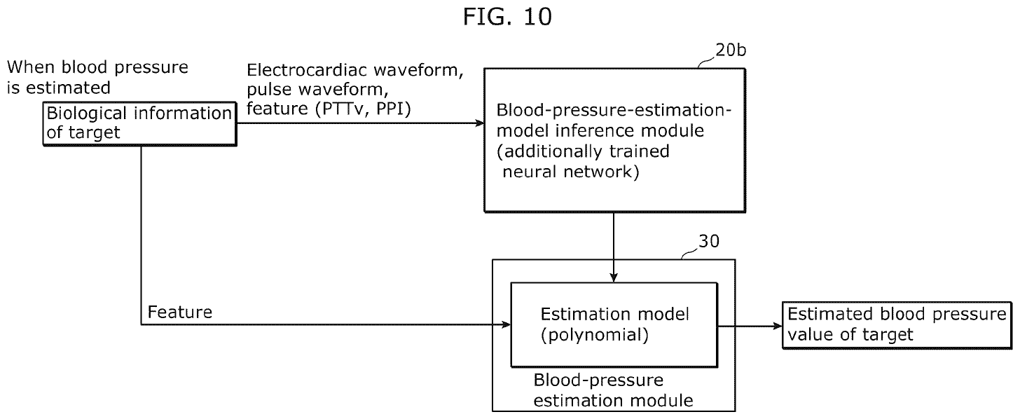

FIG. 10 is a diagram illustrating one example of operations carried out by blood pressure estimation device 100b according to Embodiment 2 when blood pressure is estimated.

The use of the above-described additionally trained neural network personalized for the target by blood-pressure-estimation-model inference module 20b enables blood-pressure-estimation-model inference module 20b to output, from an electrocardiac waveform, a pulse waveform, and features such as a PTTv and a PPI of the target, a coefficient and a constant (blood-pressure estimation module 30) for an estimation model even more suitable for the target. Thereafter, a blood pressure value of the target can be even more accurately estimated by multiplying the coefficient by a feature (e.g., features x₁ through x₂₅) of the target and adding the constant.

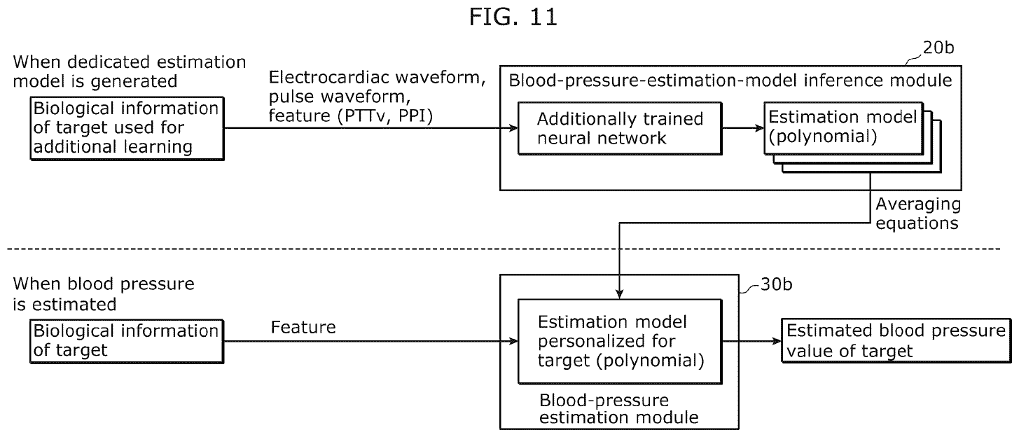

For example, although FIG. 10 shows an example in which an estimation model is inferred every time a blood pressure value of the target is estimated, an estimation model personalized for the target may be generated when the target uses blood pressure estimation device 100b for the first time, and from then on, the estimation model personalized for the target may be used when a blood pressure value of the target is estimated. The foregoing will be described with reference to FIG. 11.

FIG. 11 is a diagram illustrating one example of operations carried out by blood pressure estimation device 100b according to Embodiment 2 when blood pressure is estimated. In the example, blood pressure estimation device 100b includes, instead of blood-pressure estimation module 30, blood-pressure estimation module 30b that estimates a blood pressure value of a target using an estimation model personalized for the target.

Blood-pressure-estimation-model inference module 20b inputs, into a trained model (additionally trained neural network) personalized for the target, several items (e.g., three items) of biological information (e.g., an electrocardiac waveform, a pulse waveform, a PTTv, and a PPI) which were used when the additional machine learning was performed, to infer several (e.g., three) blood pressure value estimation models for the target. Thereafter, blood-pressure-estimation-model inference module 20b generates one estimation model personalized for the target out of the several inferred blood pressure value estimation models for the target. For example, blood-pressure-estimation-model inference module 20b generates the one estimation model personalized for the target by obtaining the average value of coefficients and constants of the several inferred blood pressure value estimation models (polynomials) for the target. Note that when the average value is obtained, the average value may be obtained excluding an outlier. In addition, the median value may be obtained instead of the average value to generate an estimation model personalized for the target.

Thereafter, blood-pressure estimation module 30b estimates a blood pressure value of the target, based on the obtained biological information (e.g., features x₁ through x₂₅) of the target and the inferred estimation model personalized for the target. As described, an estimation model need not be inferred every time a blood pressure value of the target is estimated, and after an estimation model personalized for the target is generated, a blood pressure value of the target can be estimated using the estimation model personalized for the target.

As has been described above, since blood pressure values and items of biological information are used when a trained model is generated, the generated trained model can be additionally trained using biological information and a blood pressure value of the target. Specifically, blood pressure values of a plurality of people are also used as training data, and machine learning is performed in advance to reduce differences between estimated blood pressure values of the plurality of people which are estimated by estimation models and the blood pressure values used as the training data. Accordingly, additional learning using biological information and a blood pressure value of the target can be performed. This additional machine learning performed using the biological information of the target as input data and the blood pressure value of the target as training data can generate a trained model personalized for the target that outputs an estimation model that can accurately estimate a blood pressure value of the target.

Note that, as well in Embodiment 2, learning module 200b may be provided in an external computer outside blood pressure estimation device 100b in the same manner as the variation of Embodiment 1.

### Embodiment 3

Next, a blood pressure estimation device according to Embodiment 3 will be described.

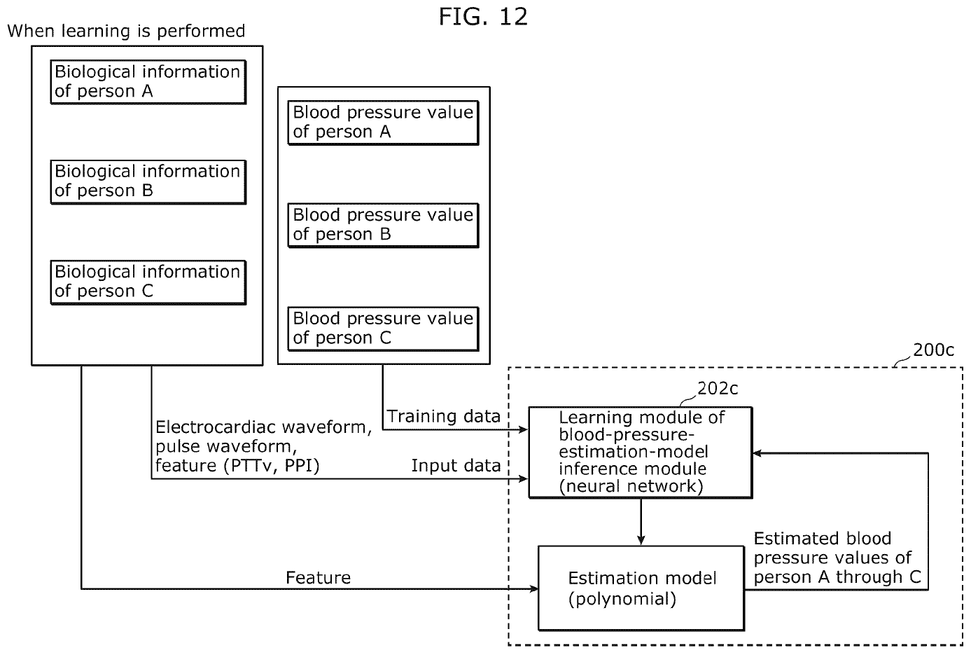

FIG. 12 is a diagram illustrating one example of operations carried out by the blood pressure estimation device according to Embodiment 3 when learning is performed.

The blood pressure estimation device according to Embodiment 3 is different from blood pressure estimation device 100b according to Embodiment 2, in that the blood pressure estimation device includes learning module 200c instead of learning module 200b. Other than the above, the blood pressure estimation device is the same as the blood pressure estimation device according to Embodiment 2, and thus detailed description will be omitted.

As illustrated in FIG. 12, learning module 200c (learning module of blood-pressure-estimation-model inference module 202c) performs machine learning that uses, as input data, items of biological information of a plurality of people and, as training data, blood pressure values of the plurality of people to generate a trained model. Unlike learning module 200b according to Embodiment 2, learning module 200c does not include blood pressure estimation model generator 201. In other words, learning module of blood-pressure-estimation-model inference module 202c does not use, as training data, blood pressure value estimation models for the plurality of people. Even in this case, learning module of blood-pressure-estimation-model inference module 202c learns a neural network that outputs a blood pressure value estimation model based the items of biological information of the plurality of people to generate a trained neural network model (trained model).

Specifically, learning module of blood-pressure-estimation-model inference module 202c learns a neural network that uses, as input data, electrocardiac waveforms, pulse waveforms, PTTvs, and PPIs of the plurality of people (e.g., person A, person B, and person C) and, as training data, blood pressure values of the plurality of people (e.g., person A, person B, and person C) to output a blood pressure value estimation model (polynomial). Every time learning module of blood-pressure-estimation-model inference module 202c performs learning, or stated differently, every time a weighting factor of the neutral network is updated, learning module of blood-pressure-estimation-model inference module 202c infers an estimation model based on the input data and, inputs features (e.g., features x₁ through x₂₅) into the inferred estimation model to estimate blood pressure values of the plurality of people (e.g., person A, person B, and person C). Learning module of blood-pressure-estimation-model inference module 202c compares the estimated blood pressure values with the blood pressure values that are the training data to obtain differences, and performs learning so as to reduce these differences. As described above, learning module of blood-pressure-estimation-model inference module 202c generates a trained model.

Since, as well in Embodiment 3, machine learning using items of biological information as input data and blood pressure values as training data is performed when a trained model is generated, additional learning using biological information of a target as input data and a blood pressure value of the target as training data can be performed.

Note that, as well in Embodiment 3, learning module 200c may be provided in an external computer outside the blood pressure estimation device in the same manner as the variation of Embodiment 1.

### OTHER EMBODIMENTS

Hereinbefore, the blood pressure estimation device according to one or more aspects of the present disclosure has been described based on the embodiments, but the present disclosure is not limited to these embodiments. The scope of the one or more aspects of the present disclosure may encompass embodiments as a result of making, to the embodiments, various modifications that may be conceived by those skilled in the art and combining elements in different embodiments, as long as the resultant embodiments do not depart from the spirit of the present disclosure.

For example, although the above-described embodiments have presented an example in which multiple regression analysis is used by blood pressure estimation model generator 201, a neural network may be used. For example, blood pressure estimation model generator 201 may generate, as an estimation model for each of a plurality of people, a weighting factor of the neural network. Moreover, learning module of blood-pressure-estimation-model inference module 202 may learn a neural network that uses, as input data, electrocardiac waveforms, pulse waveforms, PTTvs, and PPIs of the plurality of people and, as training data, generated weighting factors of the neural network of the plurality of people, to output a blood pressure value estimation model (neural network). In this case, blood-pressure-estimation-model inference module 20 infers, as a blood pressure value estimation model for the target, a weighting factor of the neural network that uses obtained biological information (e.g., features x₁ through x₂₅) of the target as an input.

For example, the above-described embodiments have presented, as an example of biological information that is to be used by the blood-pressure-estimation-model inference module to infer an estimation model, an electrocardiac waveform, a pulse waveform, and information (e.g., a PTTv and a PPI) for conforming a time period of the electrocardiac waveform to a time period of the pulse waveform, and, as an example of biological information that is to be used by the blood-pressure estimation module to estimate a blood pressure value, features (e.g., features x₁ through x₂₅) pertaining to the heart's electrical activity and a pulse wave. In other words, the above-described embodiments have presented an example in which the biological information used by the blood-pressure-estimation-model inference module to infer an estimation model and the biological information used by the blood-pressure estimation module to estimate a blood pressure value are different. However, the biological information used by the blood-pressure-estimation-model inference module to infer an estimation model and the biological information used by the blood-pressure estimation module to estimate a blood pressure value may be the same. For example, both the biological information used by the blood-pressure-estimation-model inference module to infer an estimation model and the biological information used by the blood-pressure estimation module to estimate a blood pressure value may be features (e.g., features x₁ through x₂₅) pertaining to the heart's electrical activity and a pulse wave.

For example, although the above-described embodiments have presented an example in which the information for conforming a time period of an electrocardiac waveform to a time period of a pulse waveform includes a PTTv and a PPI, the information is not limited by the foregoing. For example, the information for conforming a time period of an electrocardiac waveform to a time period of a pulse waveform may include a pulse transmission time (PTTv) from an R wave of the electrocardiac waveform to a peak of the pulse waveform and an R-R interval (RRI) from the R wave of the electrocardiac waveform to the next R wave of the electrocardiac waveform.

For example, although the above-described embodiments have presented an example in which biological information is information pertaining to the heart's electrical activity and a pulse wave, the biological information is not limited to the foregoing. For example, the biological information may be a ballistocardiogram, phonocardiogram, bioimpedance, or the like. Furthermore, the biological information, such as the height, weight, saturation of percutaneous oxygen (SpO2), may be used for an estimation of a blood pressure value.

For example, the present disclosure can be implemented not only as a blood pressure estimation device, but also as a blood pressure estimation method including steps (processes) performed by the elements included in the blood pressure estimation device.

The blood pressure estimation method is a blood pressure estimation method to be executed by a computer and includes, as illustrated in FIG. 3: an obtaining step (step S11) obtaining biological information of a target; a blood pressure estimation model inferencing step (step S12) inputting the biological information of the target which has been obtained into a trained model generated through machine learning, and inferring a blood pressure value estimation model for the target; and blood pressure estimation step (step S13) estimating a blood pressure value of the target based on the biological information of the target which has been obtained and the blood pressure value estimation model for the target which has been inferred.

For example, the present disclosure can implement these steps included in the blood pressure estimation method as a program to be executed by a processor. Furthermore, the present disclosure can be implemented as a non-transitory computer-readable recording medium, such as a CD-ROM, on which the program is recorded.

For example, when the present disclosure is implemented by a program (software), each of the steps is performed by execution of the program using hardware resources, such as a central processing unit (CPU), memory, and an input/output circuit. In other words, each step is performed by the CPU performing arithmetic operation by obtaining data from, for example, the memory or the input/output circuit, and outputting results of the arithmetic operation to, for example, the memory or the input/output circuit.

Note that in the above-described embodiments, each of the elements included in the blood pressure estimation device may be implemented by executing a software program suitable for the element. Each element may be implemented as a result of a program execution unit, such as a central processing unit (CPU), processor, or the like, loading and executing a software program stored in a storage medium such as a hard disk or a semiconductor memory.

Some of or all of functions of the blood pressure estimation device according to the above-described embodiments are typically implemented as an LSI circuit, which is an integrated circuit. Each of these functions may be individually implemented as a single chip, or some or all of the functions may be implemented as a single chip. Moreover, circuit integration is not limited to LSI; functions may be implemented as a dedicated circuit or generic processor. A field programmable gate array (FPGA) that is programmable after manufacturing of the LSI circuit, or a reconfigurable processor whose circuit cell connections and settings in the LSI circuit are reconfigurable, may be used.

Although only some exemplary embodiments of the present disclosure have been described in detail above, those skilled in the art will readily appreciate that many modifications are possible in the exemplary embodiments without materially departing from the novel teachings and advantages of the present disclosure. Accordingly, all such modifications are intended to be included within the scope of the present disclosure.

### INDUSTRIAL APPLICABILITY

The present disclosure is applicable to, for example, devices such as wearable devices that measure blood pressure without using a cuff.

1. A blood pressure estimation device comprising:
a sensing module that obtains biological information of a target;
a blood-pressure-estimation-model inference module that inputs the biological information of the target which has been obtained into a trained model generated through machine learning to infer a blood pressure value estimation model for the target; and
a blood-pressure estimation module that estimates a blood pressure value of the target based on the biological information of the target which has been obtained and the blood pressure value estimation model for the target which has been inferred.

2. The blood pressure estimation device according to claim 1, further comprising:
a learning module that performs the machine learning based on items of biological information of a plurality of people to generate the trained model.

3. The blood pressure estimation device according to claim 2, wherein
the learning module performs the machine learning based on blood pressure values and the items of biological information of the plurality of people to generate the trained model.

4. The blood pressure estimation device according to claim 3, wherein
the learning module performs the machine learning using the items of biological information of the plurality of people as input data and the blood pressure values of the plurality of people as training data, to generate the trained model.

5. The blood pressure estimation device according to claim 3, wherein
the learning module:
for each person in the plurality of people, generates a blood pressure value estimation model for the person, based on the blood pressure value of the person and the biological information of the person; and
performs the machine learning using the items of biological information of the plurality of people as input data and the blood pressure value estimation models for the plurality of people which have been generated and the blood pressure values of the plurality of people as training data, to generate the trained model.

6. The blood pressure estimation device according to claim 3, wherein
the learning module performs additional machine learning using the biological information of the target which has been obtained as input data and a blood pressure value of the target as training data, to generate the trained model personalized for the target.

7. The blood pressure estimation device according to claim 6, wherein
the blood-pressure-estimation-model inference module inputs, into the trained model personalized for the target, items of biological information of the target each of which is the biological information of the target used when the additional machine learning was performed to infer a plurality of blood pressure value estimation models for the target each of which is the blood pressure value estimation model for the target, and generates one estimation model personalized for the target from the plurality of blood pressure value estimation models for the target which have been inferred, and
the blood-pressure estimation module estimates the blood pressure value of the target, based on the biological information of the target which has been obtained and the one estimation model personalized for the target.

8. The blood pressure estimation device according to claim 2, wherein
the learning module:
for each person in the plurality of people, generates a blood pressure value estimation model for the person, based on a blood pressure value of the person and the item of biological information of the person; and
performs the machine learning based on the items of biological information of the plurality of people and the blood pressure value estimation models for the plurality of people which have been generated, to generate the trained model.

9. The blood pressure estimation device according to claim 5, wherein
the learning module generates the blood pressure value estimation models for the plurality of people through regression analysis.

10. The blood pressure estimation device according to claim 1, wherein
the blood-pressure-estimation-model inference module infers, as the blood pressure value estimation model for the target, a polynomial that uses the biological information of the target which has been obtained as a variable.

11. The blood pressure estimation device according to claim 1, wherein
the blood-pressure-estimation-model inference module infers, as the blood pressure value estimation model for the target, a weighting factor of a neural network that uses the biological information of the target which has been obtained as an input.

12. The blood pressure estimation device according to claim 1, wherein
the biological information pertains to heart's electrical activity and a pulse wave.

13. The blood pressure estimation device according to claim 12, wherein
the blood-pressure-estimation-model inference module inputs, as the biological information of the target which has been obtained, an electrocardiac waveform, a pulse waveform, and information for conforming a time period of the electrocardiac waveform to a time period of the pulse waveform into the trained model, to infer the blood pressure value estimation model for the target, and
the blood-pressure estimation module estimates the blood pressure value of the target, based on a feature pertaining to the heart's electrical activity and the pulse wave as the biological information of the target which has been obtained and the blood pressure value estimation model for the target which has been inferred.

14. The blood pressure estimation device according to claim 13, wherein
the information for conforming the time period of the electrocardiac waveform to the time period of the pulse waveform includes a pulse transmission time from an R wave of the electrocardiac waveform to a valley of the pulse waveform and a time period from a peak of the pulse waveform to a next peak of the pulse waveform.

15. The blood pressure estimation device according to claim 1, wherein
the blood-pressure estimation module multiplies the biological information of the target which has been obtained by a coefficient included in the blood pressure value estimation model for the target which has been inferred, to estimate the blood pressure value of the target.

16. A blood pressure estimation method to be executed by a computer, the blood pressure estimation method comprising:
obtaining biological information of a target;
inputting the biological information of the target which has been obtained into a trained model generated through machine learning, and inferring a blood pressure value estimation model for the target; and
estimating a blood pressure value of the target based on the biological information of the target which has been obtained and the blood pressure value estimation model for the target which has been inferred.

17. A non-transitory computer-readable recording medium for use in a computer, the recording medium having recorded thereon a computer program for causing the computer to execute the blood pressure estimation method according to claim 16.
